(19) United States
(12) Patent Application Publication
(10) Pub. No.: US 2023/0218570 A1
(43) Pub. Date: Jul. 13, 2023

(54) Methods and Compositions for Treating Mild Traumatic Brain Injury, Post-Traumatic Stress Disorder and Mild Traumatic Brain Injury with Post Traumatic Stress Disorder

(71) Applicant: LOBE SCIENCES LTD., Vancouver (CA)

(72) Inventor: Maghsoud DARIANI, Fanwood, NJ (US)

(21) Appl. No.: 18/009,161

(22) PCT Filed: Jun. 15, 2021
(86) PCT No.: PCT/US2021/037319

§ 371 Date: Dec. 8, 2022

Related U.S. Application Data
(60) Provisional application No. 63/040,032, filed on Jun. 17, 2020.

(51) Int. Cl. A61K 31/36; A61K 31/198; A61P 25/00

(52) U.S. Cl. CPC A61K 31/36; A61K 31/198; A61P 25/00

(57) ABSTRACT

Methods and compositions for treating mild traumatic brain injury (mTBI), post-traumatic stress disorder (PTSD) or mTBI with PTSD with an empathogen and N-acetylcysteine are provided.

This patent application claims the benefit of priority from U.S. provisional application Ser. No. 63/040,032 filed Jun. 17, 2020, teachings of which are herein incorporated by reference in their entirety.

### FIELD

The present invention relates to methods for treating or alleviating symptoms of mild traumatic brain injury, post-traumatic stress disorder (PTSD) and mild traumatic brain injury (mTBI) with PTSD via administration of an empathogen in combination with N-acetylcysteine (NAC). Compositions comprising MDMA in combination with NAC for use in treating or alleviating symptoms of mTBI, PTSD and mTBI with PTSD are also provided.

### BACKGROUND

Post-traumatic stress disorder (PTSD) and traumatic brain injury (TBI) often coexist because brain injuries are often sustained in traumatic experiences (Bryant, R. Dialogues Clin Neurosci. 2011 September; 13(3): 251-262).

TBI involves damage to the brain from an external force. Brain injuries can involve contusion, brain laceration, intracranial hematoma, contrecoup injury, shearing of nerve fibers, intracranial hypertension, hypoxia, anemia, metabolic anomalies, hydrocephalus, and subarachnoid hemorrhage (Bryant, R. Dialogues Clin Neurosci. 2011 September; 13(3): 251-262). Severity of TBI is typically described in terms of mild or moderate/severe with mild traumatic brain injury (mTBI) usually being defined as: (i) an external injury to the brain; (ii) confusion, disorientation, or loss of consciousness for 30 minutes or less; (iii) Glasgow Coma Scale score of 13 to 15; and (iv) post-traumatic amnesia for less than 24 hours (American Congress of Rehabilitation Medicine. Definition of mild traumatic brain injury. J Head Trauma Rehab. 1993; 8:86-87; Carroll et al. J Rehab Med. 2004 36:113-125; Ruff et al. Arch Clin Neuropsychol. 2009; 24:3-10).

PTSD reactions can be immediate or longer-term and are distinguished diagnostically because acute stress reactions are frequent, but often transient, as compared to the less common persistent PTSD responses. In terms of the persistent responses, PTSD is described in the American Psychiatric Association's DSM-IV as an anxiety disorder that comprises five major criteria (American Psychiatric Association. Diagnostic and Statistical Manual of Mental Disorders. 4th ed. Washington, D.C.: American Psychiatric Association. 1994). First, one must have been exposed to or witness an event that is threatening to safety, and one must respond to this event with fear, horror, or helplessness. Second, one must report a re-experiencing symptom, which may include intrusive memories, nightmares, a sense of reliving the trauma, or psychological or physiological distress when reminded of the trauma. Third, there need to be at least three avoidance symptoms, which can include active avoidance of thoughts, feelings, or reminders of the trauma, inability to recall some aspect of the trauma, withdrawal from others, or emotional numbing. Fourth, one must suffer marked arousal, which can include insomnia, irritability, difficulty concentrating, hypervigilance, or heightened startle response. Finally, these symptoms must cause marked impairment to one's functioning, and can only be diagnosed when they are present at least 1 month after the trauma.

It was previously argued that PTSD could not develop following TBI because the impaired consciousness at the time of trauma precluded encoding of the traumatic experience, and this prevented trauma memories that are necessary for PTSD development (Sbordone R J., Liter J C. Brain Inj. 1995; 9:405-412; Price K P Law J. 1994; 43:113-120). More recently, however, evidence has accumulated that PTSD can develop following mild TBI (Bryant R A., Harvey A G. Am J Psychiatry. 1998; 155:625-629; Middelboe et al. Eur Psychiatry. 1992; 7:183-189; Ohry et al. Brain inj. 1996; 10:687-695; Hickling et al. Brain Inj. 1998; 12:265-274; Castro C A., Gaylord K M. J Trauma-Inj Infect Crit Care. 2008; 64:S205-S206; Greenspan et al. Brain Inj. 2006; 20:733-742; Harvey A G., Bryant R A. Am J Psychiatry. 2000; 157:626-628; Hoge et al. N Engl J Med. 2008; 358:453-463; Levin et al. J Clin Exp Neuropsychol. 2001; 23:754-769.

Several models have been set forth to explain how PTSD can develop following TBI including fear conditioning, memory reconstruction and postamnesia resolution (Bryant, R. Dialogues Clin Neurosci. 2011 September; 13(3): 251-262).

The definitions of post concussive syndrome (PCS) can vary, but generally overlap somewhat with symptoms of PTSD. For example, the International Classification of Diseases (ICD-10) stipulates that PCS is defined by headaches, dizziness, general malaise, fatigue, noise intolerance, irritability, emotional lability, depression, or anxiety, concentration or memory difficulty, sleep disturbance, reduced tolerance to alcohol, and a preoccupation with these symptoms and fear of permanent brain damage (World Health Organization. The ICD-10 Classification of Mental and Behavioural Disorders: Clinical Descriptions and Diagnostic Guidelines. 1995 éd. Geneva, Switzerland: World Health Organization. 1995). The Appendix of the DSM.-IV describes PCS as fatigue, sleep disturbance, headaches, dizziness, irritability, anxiety or depression, changes in personality, and apathy (American Psychiatric Association. Diagnostic and Statistical Manual of Mental Disorders. 4th ed. Washington, D.C.: American Psychiatric Association. 1994). These descriptions clearly overlap with common symptoms of post-traumatic stress.

N-acetylcysteine (also known as acetylcysteine or N-acetyl-L-cysteine or NAC) is primarily used as a mucolytic agent and in the management of acetaminophen poisoning. It is a derivative of cysteine with an acetyl group attached to the amino group of cysteine. NAC is essentially a prodrug that is converted to cysteine (in the intestine by the enzyme aminoacylase 1) and absorbed in the intestine into the blood stream. Cysteine is a key constituent to glutathione which is an antioxidant capable of preventing damage to important cellular components caused by reactive oxygen species such as free radicals, peroxides and lipid peroxides. Hence administration of N-acetylcysteine replenishes glutathione levels in the body which can help mitigate symptoms for a variety of diseases exacerbated by reactive oxygen species (ROS). For instance, N-acetylcysteine is commonly used in individuals with renal impairment to prevent the precipitation of acute renal failure. N-acetylcysteine has been shown to have efficacy in treating mild to moderate traumatic brain injury including ischemic brain injury, particularly in reducing neuronal losses, and also reducing cognitive and neurological symptoms when administered promptly after injury. N-acetylcysteine is also being successfully used to treat a variety of neuropsychiatric and neurodegenerative disorders including cocaine, cannabis, and smoking addictions, Alzheimer's and Parkinson's diseases, autism, compulsive and grooming disorders, schizophrenia, depression, and bipolar disorder.

The empathogen 3,4-methylenedioxy-methamphetamine (MDMA) is a drug that acts as both a stimulant and a hallucinogen, producing feelings of increased energy, elevated mood and enhanced sense of well being, pleasure and euphoria, trust and emotional closeness resulting in willingness to discuss emotionally charged memories, increased extroversion, empathy towards others and enhanced sensory and time perception. These effects of empathogens such as MDMA make them excellent therapeutics to be used in conjunction with psychotherapy for the treatment of neuropsychiatric disorders such as depression, anxiety, obsessive compulsive disorder, and PTSD where open and uninhibited discussion with the therapist is essential for successful treatment.

The mechanism of action of empathogens such as MDMA involves three neurotransmitters: i) increased serotonin in the brain, resulting in euphoria, empathy, trust and emotional warmth towards others, elevated mood and sensory perception, and sexual arousal; ii) increased dopamine in the brain, leading to feelings of pleasure, euphoria and increased energy; and iii) increased norepinephrine, leading to stimulant effects such as increased heart rate and blood pressure.

MDMA along with psychotherapy is being evaluated for treatment of PTSD by various groups. Food and Drug Administration recently approved a special protocol for phase 3 clinical trials to evaluate the efficacy of MDMA-assisted psychotherapy for the treatment of PTSD.

Administration of an empathogen in combination with NAC is expected to result in improved outcomes in alleviating symptoms associated with mTBI, PTSD and mTBI with PTSD.

### SUMMARY

An aspect of the present invention relates to a method for alleviating one or more symptoms of mTBI, PTSD or mTBI with PTSD. The method comprises administering to an individual suffering from mTBI, PTSD or mTBI with PTSD an empathogen and NAC.

Another aspect of the present invention relates to pharmaceutical formulations and kits thereof comprising an empathogen and NAC for use in alleviating one or more symptoms of mTBI, PTSD or mTBI with PTSD.

### DETAILED DESCRIPTION

The present invention provides methods and compositions for alleviating one or more symptoms of mTBI, PTSD or mTBI with PTSD.

The methods and compositions involve administration of an empathogen in combination with NAC.

N-acetylcysteine or NAC is a potent antioxidant, increasing levels of glutathione in the body, which can help protect brain cells from reactive oxygen species and trauma to the head. NAC has been shown to have efficacy in treating mild to moderate traumatic brain injury including ischemic brain injury, particularly in reducing neuronal losses, and also reducing cognitive and neurological symptoms when administered promptly after injury.

Empathogens, also referred to as entactogens, are a class of psychoactive drugs that produce experiences of emotional communion, oneness, relatedness, emotional openness—that is, empathy or sympathy. Nonlimiting examples include 3,4-methylenedioxy-methamphetamine (MDMA), 3,4-methlenedioxyamphetamine (MDA), 3,4-methylenedioxy-N-ethylamphetamine (MDEA), 3,4-methylenedioxy-N-hydroxyamphetamine (MDOH), N-methyl-1,3-benzodioxolylbutanamine (MBDB), 6-(2-aminopropyl)benzofuran (6-APB), methylone, mephedron, αMT, αET, and 5,6-methylenedioxy-2-aminoindane (MDAI). In one nonlimiting embodiment, the empathogen used in these methods and compositions is 3,4-methylenedioxy-methamphetamine or MDMA, a compound that acts as both a stimulant and a hallucinogen, producing feelings of increased energy, elevated mood and enhanced sense of well being, pleasure and euphoria, trust and emotional closeness resulting in willingness to discuss emotionally charged memories, increased extroversion, empathy towards others and enhanced sensory and time perception.

As used herein, by “alleviating one or more symptoms of mTBI, PTSD or mTBI with PTSD”, it is meant to decrease severity of one or more of intrusive memories, nightmares, a sense of reliving the trauma, or psychological or physiological distress when reminded of the trauma, active avoidance of thoughts, feelings, or reminders of the trauma, inability to recall some aspect of the trauma, withdrawal from others, or emotional numbing, insomnia, irritability, difficulty concentrating, hypervigilance, or heightened startle response. The inventors believe that the studies disclosed herein will demonstrate that the combination therapy of an empathogen and NAC will be more effective in alleviating one or more symptoms of mTBI, PTSD and/or mTBI with PTSD than either agent individually. Preferred combination therapies in accordance with this invention are synergistic, meaning better than additive in their efficacy in alleviating one or more symptoms of mTBI, PTSD and/or mTBI with PTSD.

In one nonlimiting embodiment, the empathogen and NAC are administered in combination immediately following the mTBI or within 12 to 24 hours of the mTBI. In one nonlimiting embodiment, the empathogen and NAC are administered in combination upon the onset of symptoms of PTSD. In one nonlimiting embodiment, the empathogen and NAC are administered after a traumatic event typically leading to PTSD. As will be understood by the skilled artisan upon reading this disclosure, dosages can be determined by the attending physician, according to the extent of the injury to be treated, method of administration, patient's age, weight, contraindications and the like.

As used herein, by “in combination” it is meant to include coadministration of an empathogen and NAC, sequential administration of the empathogen followed by NAC, or sequential administration of NAC followed by the empathogen.

In one nonlimiting embodiment, NAC is administered within 12 hours of the traumatic brain injury, or alternatively with 6 hours of the traumatic brain injury, or alternatively within 3 hours of the traumatic brain injury. In these embodiments, NAC may be administered as a single dose or as multiple doses.

In one nonlimiting embodiment, multiple doses of NAC are administered over a 72 hour period following the traumatic brain injury.

In one nonlimiting embodiment, NAC is administered daily or every two days until symptoms of the traumatic brain injury are alleviated.

In one nonlimiting embodiment, NAC is administered upon onset of symptoms of PTSD.

In one nonlimiting embodiment, NAC is administered within 3 to 24 hours of a traumatic event which typically results in PTSD. In this embodiment, NAC may be administered as a single dose or as multiple doses.

In one nonlimiting embodiment, multiple doses of NAC are administered over a 72 hour period following the traumatic event.

NAC may be administered by any route providing for delivery of effective amounts to the brain. Examples of routes of administration include, but are in no way limited to, intravenous, intranasal, oral, topical, transdermal or via inhalation.

Doses of NAC which have been administered safely for various conditions in humans range from 70 mg up to 6 grams per day. See webmd with the extension com/vitamins/ai/ingredientmono-1018/n-acetyl-cysteine-nac of the world wide web. As will be understood by the skilled artisan upon reading this disclosure, similar dosing regimens to those already used for NAC as well as alternative dosing regimens determined to be clinically relevant may be used.

For an empathogen such as MDMA, doses will typically range between 75-100 mg. In some embodiments, chronic daily doses may be administered. However, for the empathogen, it may be preferable to administer two to three single-dose treatments. As will be understood by the skilled artisan upon reading this disclosure, similar dosing regimens to those already used for this empathogen as well as alternative dosing regimens determined to be clinically relevant may be used.

In addition, microdosing, a practice of using sub-threshold doses (microdoses) of the empathogen may be used.

The empathogen can be administered before, simultaneously or after administration of the NAC.

In one nonlimiting embodiment, the empathogen and NAC are coadministered in a solid dosage formulation.

In one nonlimiting embodiment, the empathogen and NAC are coadministered in a nasal spray formulation.

In one nonlimiting embodiment, empathogen and NAC are coadministered by nasal spray transducer programmed time release administration. A nonlimiting device for such administration is described in PCT/US2021/028068 filed Apr. 20, 2021, teachings of which are incorporated herein by reference in their entirety.

In one nonlimiting embodiment, the empathogen and NAC are coadministered in a nasal spray where therapeutically active amounts of each are dissolved or suspended in solution or mixtures of excipients (e.g., preservatives, viscosity modifiers, emulsifiers, buffering agents) in nonpressurized dispensers that deliver a spray containing a metered dose of each ingredient.

In one nonlimiting embodiment, empathogen and NAC coadministration enables pathological memory eradication for treatment of mTBI, PTSD or mTBI with PTSD.

In one nonlimiting embodiment, empathogen and NAC coadministration is expected to prevent pathological conversion of short term memory to pathological long term memory (LTM) and promote disengagement of pathological LTM by a chemical agonist/antagonist shock similar to insulin and/or electric shock therapy. Such formulations are expected to be useful in treating disorders related to pathological LTM such as mTBI, PTSD and mTBI with PTSD.

In one nonlimiting embodiment, an encapsulation technique is used to enclose various concentrations of the empathogen and NAG in a relatively stable shell known as a capsule, allowing them to, for example, be taken orally. In one nonlimiting embodiment, the formulation of the present invention comprises a hard-shelled capsule containing dry, powdered ingredients, miniature pellets made by processes such as extrusion and spheronization or mini tablets. The hard-shelled capsules are typically made in two halves: a smaller-diameter body that is filled and then sealed using a larger-diameter cap. The capsule itself is typically made from aqueous solutions of gelling agents, such as animal protein (mainly gelatin) or plant polysaccharides or their derivatives (such as carrageenans and modified forms of starch and cellulose). Other ingredients can be added to the gelling agent solution including plasticizers such as glycerin or sorbitol to decrease the capsule's hardness, coloring agents, preservatives, disintegrants, lubricants and surface treatment.

The following nonlimiting examples are provided to further illustrate the present invention.

### EXAMPLES

### Animal Model for mTBI and PTSD

Small animal models, in particular mice and rats, are essential in the study of mTBI and PTSD. See Schöner J et al. J Cell Mol Med. 2017 (10):2248-2256; Prater et al. Neuropsychopharmacology. 2017 42(8):1706-1714; and Perez-Garcia et al. Neuropharmacology. 2019 145(Pt B):220-229. These animal models allow investigators to study the functional impact of both insults and to examine the anatomic pathologic correlates. Moreover, these animals allow investigators to include enough animals to overcome the natural heterogeneity of both disorders (mTBI and PTSD).

Rats provide an excellent model to study changes in behavior since rats are amenable to the training necessary to display the characteristic responses of PTSD (which involves changes in behavior of a previous trained and reliable model behavior). Further, rats are hardier and a better model for the dual insult of mTBI and PTSD.

### Materials and Methods Experimental Design

Five main exposure groups are examined as follows: 1) No exposure, 2) Sham fluid percussion (surgical prep but no fluid percussion injury) plus PTSD trigger, 3) FP plus PTSD trigger, 4) Blast plus PTSD trigger, 5) Repeated Blast (known to be a PTSD trigger)alone. Each exposure is detailed below. In each of the 5 groups there will be four dosing paradigms as follows A) Vehicle alone, NAC alone, empathogen alone and D) NAC plus empathogen. Preferred is that 12-15 rats are examined per group. However, as will be understood by the skilled artisan upon reading this studies, positive results from smaller groups are also demonstrative of efficacy. Comparisons are made between the performance of the rats within each group on each test using stated statistical methods to assess group mean differences (ANOVA, etc.)

### Methodology in Detail

Gavage—A powder comprising a combination of NAC and the empathogen is solubilized in sterile water. The aqueous solution is then given orally by gavage to the animals once daily for seven days beginning within one hour of exposure and continuing for six more daily doses. Doses administered will be 1 ml of each per gavage (equivalent to ˜3 to 10 mg/kg total of empathogen and 20 mg/kg total of NAC per gavage per animal).

Production of mTBI—Two mTBT models are utilized for this experiment: a fluid percussion model (mild—moderate mTBI) and a blast model (mild mTBI)

Fluid Percussion Model

Day 1: For surgical preparation for the injury cap, isoflurane anesthesia is maintained via nose cone and the injury cap is placed on the exposed dura as follows. The rat's head is shaved and swabbed with clorohexadine solution. The rat is then placed in a stereotaxic frame and the scalp surgically incised. A parasagittal craniotomy (4.8 mm) using a trephine is performed at 3.8 mm posterior to bregma and 2.5 mm lateral to the midline. A sterile plastic injury tube (the plastic connector of a sterile needle cut 1 cm in length and trimmed to fill the craniotomy perfectly) is next placed over the exposed dura and bonded by crynoacrylic adhesive to the skull. Dental acrylic is then poured around the injury tube to obtain a perfect seal. After the acrylic has hardened, the scalp is stapled/sutured back. Animals are removed from the anesthesia and returned to their home cage.

Day 2: 24 hours after the previous injury cap preparation, the rats are reanesthetized with 0.5-5% isoflurane via a custom built anesthesia chamber, the animal is placed on the table and anesthesia is administered via a nose cone until catheters are placed and the animals is intubated. A catheter is placed in the right femoral artery or tail artery to monitor arterial blood pressure and blood gases. Brain temperature is indirectly measured by a thermistor placed in the left temporalis muscle and maintained at a normothermic (37° C.) level prior and subsequent to TBI.

Rectal temperature is also maintained at normothermic levels. After intubation, the animal is connected to a respirator and ventilated with 0.5-5% isoflurane in a mixture of 70% nitrous oxide and 30% oxygen. 14G IV catheters are used for the ventilation tube which is modified to an appropriate length. The ventilation rate is 48 to 58 strokes per minute and the tidal volume is 2.5-3.5 and adjusted for the weight of the animal. The animal is paralyzed with rocuronium or pancuronium or vencuronium for mechanical ventilation to maintain arterial blood gases within normal limits. The fluid percussion device consists of a plexiglass cylindrical reservoir bounded at one end by a rubber-covered plexiglass piston with the opposite end fitted with a transducer housing and a central injury screw adapted for the rat's skull. The entire system is filled with isotonic saline. The (aseptic) metal injury screw is next firmly connected to the plastic injury tube of the intubated and anesthetized rat. The injury is induced by the descent of a metal pendulum striking the piston, thereby injecting a small volume of saline epidurally into the closed cranial cavity and producing a brief displacement (18 msec) of neural tissue. The amplitude of the resulting pressure pulse is measured in atmospheres by a pressure transducer and recorded on a PowerLab chart recording system. Sham animals undergo all surgical procedures but are not subjected to the fluid percussion pulse. In the experiments, a moderate (1.8-2.2 atm) injury is studied. Animals receive Buprenorphine after the TBI. After either the TBI or sham injury, the injury cap is removed and the scalp is closed using staples. The area around the femoral artery is prepped for sterility. The sterile incision for femoral artery cannulations is stapled as well. For tail artery incisions, the tail is sutured together with sterile sutures. After 45 min-1.5 hours, the animal awakens and is moved to an individual cage supplied with food and water until termination of the study. If the animal has difficulty eating, then the animal is humanely euthanized. The rats (pre- and post-injury) in this experiment are fed per the manufacturer's recommended daily amount of 6 pellets per day for rats. Staples or sutures are removed 10-14 days post-injury after briefly placing the animal under isoflurane anesthesia.

Blast Injury

All animals are anesthetized and placed in an animal holding tube inserted and secured one-foot within the end of the condensing tube. The animal holding tube positions the animal with the rat's dorsal head surface to the on-coming shock wave. Subjects are positioned 10 feet from the tube film diaphragm and receive a BOP wave in a head-on orientation. The holding tube allows for isoflurane gas to feed to the animal to induce anesthesia allowing exposures to live but anesthetized animals. BOP waves are measured and displayed for peak intensities, rise time and BOP wave durations using a Pacific Instruments 6000 DAQ with up to 32 channels, each with 250 kHz recording speed along with Dytran pressure transducers rated for 0 50 PSI measurement range and electronic conditioners interfaced with computers. An exposure consists of anesthetized animals receiving a single blast wave exposure. Investigations examine the effects of single 10-20 psi (Friedlander wave with overpressure-underpressure sequence) which have been shown to demonstrate pathological effects.

Production of PTSD Predatory Threat

Rats are moved to special plastic cages which contain male cat urine for 10 minutes. This exposure creates a lasting PTSD phenotype in a humane fashion (See Goswami et al. Front Behav Neurosci. 2012 6:26). This cat urine exposure takes place prior to any TBI insult.

Repeated Blast Model

A body of work has shown that repeated exposure of anesthetized rats to low level blast produces a PTSD Phenotype (See Perez-Garcia et al. Neuropharmacology. 2019 145(Pt B):220-229). In order to produce this effect rats are exposed to blast as was described earlier. This blast is repeated for three consecutive days. This PTSD model is first performed on one separate group of animals. This exposure produces both an mTBI and PTSD phenotype and therefore does not have to be combined with any other exposure.

### Outcome Measures

A variety of outcome measures are performed on all of the animals in this experiment. All outcome measures have been shown to be sensitive to changes that occur after mTBI, PTSD, and both disorders.

Auditory Startle Response

In this outcome measure, a special Plexiglas soundproof tube attached to an accelerometer and a special auditory speaker system is used. (SR labs, San Diego Calif. USA; See Pooley et al. Biol Sex Differ. 2018 9(1):32). The device is calibrated at regular intervals to measure sound levels. Rats with no pre-training are placed in the tube and given 5 minutes to acclimatize with 68 dB background white noise. After five minutes the rats are exposed to a 50 ms of 110 dB tone delivered every 30 seconds for 15 minutes. Peak whole body startle response is measured every 1 ms for 100 ms after the startle exposure in an automated fashion. The average peak value per rat is normalized by body weight to obtain a value.

Light-Dark Emergence Tasks

A light dark emergence task is performed by placing rats in a specially designed box/chamber that has a dark and lighted side separated by a tunnel. Rats naturally seek the lighted side. See Perez-Garcia et al. (2018). In this experiment rats are placed in the specially designed box for 5 minutes. The rats are placed initially in the dark side and three outcomes are measured 1) Amount of time in seconds required to reach lighted side, 2) Number of the rat entries into the lighted side, 3) Total amount of time spent in the lighted side. There are no special preparations required to perform this test and no training is required.

Sensorimotor Testing

Spontaneous Forelimb Use: This test, described by Schallert and Lindner (Can J Psychol. 1990 44(2):276-292), assesses forelimb use during voluntary, spontaneous activity by evaluating the propensity of animals to adduct their forelimbs while rearing or standing. Animals are videotaped in a clear plastic cylinder for 5 minutes. The videotapes are scored in terms of forelimb-use asymmetry during vertical movements along the wall of the cylinder and for landings after a rear: (a) independent use of the left or right forelimb for contacting the wall of the cylinder during a full rear, to initiate a weight-shifting movement or to regain center of gravity while moving laterally in a vertical posture along the wall; Wall lands/movements and floor lands are each expressed in terms of (a) percent use of the ipsilateral (non-impaired) forelimb relative to the total number of ipsilateral and contralateral placements. During a rear, the first limb to contact the wall with clear weight support (without the other limb contacting the wall within 0.5 sec) is scored as an independent wall placement for that limb. Limb use ratio is calculated as contralateral/(ipsilateral+contralateral). This is assessed prior to brain injury as well as approximately 1 week post-trauma.

Cognitive Testing

The analysis of cognitive function involves an assessment of spatial navigation using the water maze. Experiments that are primarily directed at assessing the activity of animals at numerous time points following TBI (such as when assessing the efficacy of therapeutic treatments designed to lessen the consequences of TBI) rely primarily on “acquisition” paradigms involving the simple place task and working memory task, in which the animals are required to learn a new platform location during each test session. This protocol does not involve pretraining or testing in the water maze prior to surgery.

General Procedures: The water maze used is a round pool (122 cm diameter; 60 cm deep) filled with water at 25° C. The maze is located in a quiet, windowless room, with a variety of distinct, extramaze cues. Four points on the rim designated as north (N), east (E), south (S), and west (W), serve as starting positions and divide the maze into four quadrants. A round platform is placed 1.5 cm beneath the surface of the water, at a location that varies according to the requirements of the task (see below) . The animal's movement is videotaped with a CCD video which records the swim path. The animal's swim path is then analyzed with Ethovision (Noldus) software program. This program determines path length, latency to reach the platform, time spent in each quadrant of the water maze, and swim speed.

Hidden Platform Task: The platform is located in a target quadrant of the maze. Each animal receives four trials each day that may last up to 60 seconds. If the rat successfully locates the platform within the 60 seconds, it is allowed to remain for 10 seconds. Otherwise, once 60 seconds elapses, it is placed on the platform for a period of 10 seconds. Inter-trial intervals are two to four minutes, during which rats are placed under a heat lamp.

Probe Trial: This consists of removing the platform completely from the pool. The animal is released from a predetermined position and the swim pattern is recorded for 30 seconds. An animal with intact spatial memory should spend a majority of time swimming in the target quadrant that previously contained the hidden platform.

Working Memory Task: For the working memory task, the animal is given 60 seconds to find a submerged (non-cued) platform placed in a novel location within the pool. If the rat fails to find the platform within 60 seconds, the animal is placed on the platform for 10 seconds. This is considered Trial 1. Five seconds following Trial 1, a second identical trial is conducted for that same rat. Rats are placed under a heat lamp for 4 minutes between each paired trial. After running the group of rats as above, the platform is then moved to another novel location within the pool, and the paired trials are repeated. Five paired trials occur each day for 2 days.

Auditory Brainstem Response (ABR): Hearing thresholds are determined by auditory brainstem response (ABR) via subcutaneous platinum needle electrodes placed at the vertex (reference), right mastoid (negative) and the left hind limb with the animals anesthetized with ketamine (150 mg/kg) and xylazine (10 mg/kg). Digitally-generated stimuli consist of 1024 specific frequency tone bursts at between 3 and 30 kHz with a trapezoid envelop of 5 ms overall duration. The trapezoid is presented at a 3 ms plateau with 1 ms rise and fall. The stimulus is routed through a computer-controlled attenuator to an insert earphone (Etymotic Research ER-2). The sound delivery tube of the insert earphone is positioned about 5 mm from the tympanic membrane. The output of the insert earphone is calibrated by measuring the sound pressure level at a position 4-5 mm away from the tympanic membrane. The electrical response from the recording electrode is amplified (100,000×), filtered (100-3000 Hz) and fed to an A/D converter on a signal processing board in the computer. Eight hundred to twelve hundred samples are averaged at each level. Stimuli is presented at the rate of 16/sec and the stimulus level is varied in 10 dB descending steps, until threshold is reached, then a 5 dB ascending step to confirm. Threshold is defined as the mid-point between the lowest level at which a clear response is seen and the next lower level where no response is seen. ABR is determined as a reproducible wave II response.

Statistics

All outcome measures yield measurable responses. The group mean response to each outcome is compared utilizing an analysis of variance with significant differences set at p less than or equal to 0.05. Comparisons are made between groups (types of treatment) in each exposure condition (e.g. NAC/empathogen vs. control carrier after Fluid Percussion plus PTSD stress).

What is claimed is:

1. A method for alleviating one or more symptoms of mild traumatic brain injury (mTBI), post-traumatic stress disorder (PTSD) or mTBI with PTSD, said method comprising administering to a subject suffering from mTBI, PTSD or mTBI with PTSD an empathogen and N-acetylcysteine (NAC).

2. The method of claim 1 wherein the empathogen is selected from the group consisting of 3,4-methylenedioxy-methamphetamine (MDMA), 3,4-methlenedioxyamphetamine (MDA), 3,4-methylenedioxy-N-ethylamphetamine (MDEA), 3,4-methylenedioxy-N-hydroxyamphetamine (MDOH), N-methyl-1,3-benzodioxolylbutanamine (MBDB), 6-(2-aminopropyl)benzofuran (6-APB), methylone, mephedron, αNT, αET, and 5,6-methylenedioxy-2-aminoindane (MDAI).

3. The method of claim 1 wherein the empathogen is MDMA.

4. The method of claim 1 wherein the one or more symptoms alleviated is selected from intrusive memories, nightmares, a sense of reliving the trauma, or psychological or physiological distress when reminded of the trauma, active avoidance of thoughts, feelings, or reminders of the trauma, inability to recall some aspect of the trauma, withdrawal from others, or emotional numbing, insomnia, irritability, difficulty concentrating, hypervigilance and heightened startle response.

5. The method of claim 1 wherein the empathogen and NAC are administered simultaneously.

6. The method of claim 5 wherein the empathogen and NAC are formulated in a solid dosage form and administered to a patient orally.

7. The method of claim 5 wherein the empathogen and NAC are formulated as a solution or a suspension and are delivered to a patient as a nasal spray containing a metered dose of each ingredient.

8. The method of claim 1 wherein the composition is administered to prevent pathological conversion of short term memory to long term memory (LTM) and promote disengagement of pathological LTM by a chemical agonist/antagonist shock.

9. The method of claim 1 wherein the empathogen is administered before NAC.

10. The method of claim 1 wherein the empathogen is administered after NAC.

11. A pharmaceutical composition for alleviating one or more symptoms of mild traumatic brain injury (mTBI), post-traumatic stress disorder (PTSD) or mTBI with PTSD, said composition comprising an empathogen and N-acetylcysteine (NAC) and a pharmaceutically acceptable excipient.

12. The pharmaceutical composition of claim 11 in solid formulation.

13. The pharmaceutical composition of claim 11 in solution or suspension formulation.

14. The pharmaceutical composition of claim 11 wherein the empathogen is selected from the group consisting of 3,4-methylenedioxy-methamphetamine (MDMA), 3,4-methlenedioxyamphetamine (MDA), 3,4-methylenedioxy-N-ethylamphetamine (MDEA), 3,4-methylenedioxy-N-hydroxyamphetamine (MDOH), N-methyl-1,3-benzodioxolylbutanamine (MBDB), 6-(2-aminopropyl)benzofuran (6-APB), methylone, mephedron, αMT, αET, and 5,6-methylenedioxy-2-aminoindane (MDAI).

15. The pharmaceutical composition of claim 11 wherein the empathogen is MDMA.
